(19) United States
(12) Patent Application Publication
(10) Pub. No.: US 2026/0284062 A1
(43) Pub. Date: Sep. 24, 2026

(54) Injectable Drug Composition For Treating Atherosclerosis and Method For Using Same

(71) Applicant: Konstantin Pavlovich GARYAEV, Perm (RU)

(72) Inventors: Konstantin Pavlovich Garyaev, Perm (RU); Nadezhda Aleksandrovna GARIAEVA, Perm (RU); Igor Gennadievich ZAVGORODNII, Perm (RU)

(21) Appl. No.: 19/472,345

(22) PCT Filed: Apr. 5, 2023
(86) PCT No.: PCT/RU2023/000103

§ 371 Date: Oct. 3, 2025

(51) Int. Cl. A61K 31/525; A61K 9/00; A61K 31/167; A61K 31/194; A61K 31/197; A61K 31/455; A61K 38/47; A61K 47/02; A61P 9/10

(52) U.S. Cl. CPC A61K 31/525; A61K 9/0019; A61K 31/167; A61K 31/194; A61K 31/197; A61K 31/455; A61K 38/47; A61K 47/02; A61P 9/10

(57) ABSTRACT

The invention relates to the field of medicine, and more particularly to cardiology, cardiovascular surgery, neurology, surgery and clinical lymphology, and can be used for treating patients suffering from atherosclerosis of the neck vessels, the mediastinal vessels and the leg vessels. An injectable drug composition prepared ex tempore contains nicotinamide, succinic acid, riboflavin, an anaesthetic and a solvent, the anaesthetic being lidocaine 2%, and the solvent being NaCl 0.9%. The drug composition further contains an enzyme, specifically a hyaluronidase, and a metabolic modulator, specifically meldonium dihydrate. The drug composition is administered by lymphotropic interstitial injection into the lymphatic region where the arterial segment damaged by atherosclerosis is located. The drug composition is administered using a syringe pump over the course of 30-60 minutes. Lymphotropic interstitial injections are administered once a day over a period of 10-15 days. The technical result consists in reducing the effect of interstitial swelling, increasing the efficiency of interstitial transport, reducing atherosclerotic lesions and reducing blockage of the damaged artery.

### FIELD OF THE INVENTION

The invention relates to medicine, namely to cardiology, cardiovascular surgery, neurology, surgery, and clinical lymphology and can be used to treat patients with atherosclerosis of cervical vessels, mediastinal vessels, and vessels of the lower extremities.

### BACKGROUND

Atherosclerosis is a chronic inflammatory disease of arterial vessels of the muscular and muscular-elastic type. The main pathogenetic manifestations of atherosclerosis include lipid infiltration of the intima, lipid oxidation, uptake of lipids by macrophages, formation of foam cells, disruption of oncosis and apoptosis, proliferation of connective tissue in the vessel wall, leading to formation of atherosclerotic plaques that reduces the vessel lumen. When plaque stability is impaired, the risk of thrombosis increases. Furthermore, stenosis of more than 70% of the vessel lumen is considered hemodynamically significant stenosis with a high risk of ischemic complications. Atherosclerosis develops and is found in most people. According to World Health Organization (WHO) data for 2019, 16% of all deaths from diseases were due to consequences of cardiovascular diseases, and 11% were due to consequences of cerebrovascular diseases.

Depending on localization, various clinical manifestations of atherosclerosis are distinguished. Lesions of the major cerebral vessels manifest as transient ischemic attacks, dyscirculatory encephalopathies, and ischemic strokes. Atherosclerosis of coronary vessels manifests as ischemic heart disease, cardiac arrhythmias, and myocardial infarction. Atherosclerotic lesions of the arteries of the lower extremities manifests as vasogenic intermittent claudication, pain syndrome, and gangrene of the foot or lower leg.

Correction of vascular lesions includes both conservative and surgical treatment. Vascular surgery methods include endarterectomy, stenting, bypass grafting, and heart transplantation. These methods are high-technology medical interventions, not always accessible, inapplicable in cases of multiple vessel lesions, and not indicated until the stenosis of the affected vessel lumen reaches 50%.

Drug therapy includes medicinal products of various groups, namely: for correction of endothelial dysfunction and prevention of progression of the atherosclerotic process (statins, beta-adrenergic blockers, angiotensin-converting enzyme (ACE) inhibitors, low-molecular-weight heparins, heparinoids, analogues of natural prostaglandin E1); for correction of hemorheological and hemostatic properties of blood, antiplatelet agents (Aspirin, Clopidogrel, Dipyridamole, Ticlid, Cilostazol); and antiplatelet and indirect fibrinolytic agents (nicotinic acid). Oral antiplatelet therapy with acetylsalicylic acid and clopidogrel reduces the risk of myocardial infarction, ischemic stroke and peripheral thrombosis by 20-25%, but has side effects, is characterized by variability of individual response, and therefore requires in vivo laboratory monitoring of hemostasis. Hypolipidemic therapy with statin drugs, when used for at least five years, reduces the frequency of fatal outcomes from coronary heart disease (CHD) and other cardiovascular diseases by 25-40%, but does not have a rapid effect (within hours or days), and is associated with undesirable side effects.

The main drawback of existing therapy is that neither antiplatelet therapy nor hypolipidemic therapy has a plaque-reducing effect.

Use of the drug Cytoflavin, which contains succinic acid, nicotinamide, inosine, and riboflavin as active ingredients, is known in the treatment of atherosclerosis (Instructions for Medical Use of Cytoflavin. Order of the Federal Service for Supervision in Healthcare and Social Development of 23.12.2005 No. 74). Cytoflavin is administered only by intravenous drip infusion in 100.0-200.0 ml of 5% glucose solution or 0.9% sodium chloride solution. However, in patients with atherosclerotic disease of cervical, cerebral, and coronary vessels, intravenous administration of Cytoflavin in 30 infusions does not lead to a reduction in the size of atherosclerotic plaques that could be tracked by triplex scanning.

It is known that lymphotropic administration of drugs is more effective for the treatment of many diseases (Yu.I. Borodin, Institute of Lymphology and Problems of Lymphology//Bulletin of the Siberian Branch of the Russian Academy of Medical Sciences.—2001.—No. 4.—Pp. 5-11).

A method for treating patients with obliterating diseases of the arteries of the lower extremities is known (RU Patent No. 2523412, IPC: A61K 31/047, A61K 31/194, A61K 31/245, publ. 25.02.2013), in which a mixture of medicinal substances consisting of 5.0 ml of Cytoflavin and 5.0 ml of 0.5% Novocaine is administered lymphotropically by interstitial injection into the anterolateral or posterolateral surface of the upper or middle segments of the lower leg after preliminary local anesthesia of the injection site with 5.0 ml of 0.5% Novocaine.

A method for treating cerebrovascular diseases and asthenic syndrome is known (RU Patent No. 2463045, IPC: A61K 31/194, A61K31/455, A61K31/525, publ. 10.10.2012), in which a mixture of Cytoflavin and Novocaine is administered lymphotropically by interstitial injection into the posterior suboccipital region.

A method for treating cerebrovascular diseases and asthenic syndrome is known (RU Patent No. 2678967, IPC: A61K 31/194, A61K 31/525, A61K 31/455, A61P 9/10, publ. 05.02.2019), in which a mixture of medicinal substances consisting of 10.0 ml of Cytoflavin and 20.0 ml of 0.5% Novocaine is administered lymphotropically by interstitial injection into the pretracheal space through the jugular fossa or into the supraclavicular fossa after preliminary local anesthesia of the injection site with 5.0 ml of 0.5% Novocaine.

The above analogues have the following disadvantages for large-scale and safe introduction:

1. The medicinal mixture used contains 0.5% Novocaine in an amount of 5.0-10.0 ml, which, when administered into the recommended sites, causes undesirable effects: reduction of arterial pressure up to collapse, dizziness, headache, arrhythmia. Cases of intolerance to the anesthetic Novocaine are known, which makes it impossible to use the medicinal mixture in such patients. With repeated use of Novocaine, the phenomenon of intolerance may develop.

2. In the known analogues, the injection zone of the medicinal preparation is not sufficiently defined: depth of administration, the number of fasciae through which the needle should pass, and other features are not specified, which can lead to creation of a medicinal depot at an improper location, reduce efficacy, or cause undesirable side effects.

3. In the above analogues, the medicinal mixture is administered manually with a syringe, which leads to an uncontrolled and rapid rate of administration. Administration of 10.0-20.0 ml of the medicinal mixture within less than 10 minutes results in interstitial swelling and stagnation of interstitial transport and, clinically, in a feeling of discomfort and tissue distension in the patient during and after the procedure.

### SUMMARY OF THE INVENTION

The present invention provides an injectable drug composition for the treatment of vascular atherosclerosis, which replaces therapeutic agents of the prior art that, in particular, do not reduce the size of atherosclerotic plaques and are associated with adverse effects such as arterial hypotension up to collapse, dizziness, headache, arrhythmia, and intolerance.

The present invention also provides a method of administration of the injectable drug composition, which reduces interstitial swelling during injection, improves the efficiency of interstitial transport, and reduces stenosis of the artery affected by atherosclerosis.

The object of the invention is to provide an agent for anti-inflammatory therapy, for reducing lipid peroxidation, for enhancing lymphatic drainage from the arterial wall in atherosclerosis, and for reducing the size of atherosclerotic plaques and the thickness of the intima-media complex.

Another object of the invention is to develop a method of reducing the risk of complications of atherosclerosis as an alternative to hypolipidemic therapy (statins, intestinal cholesterol absorption inhibitors (inhibitors of CH absorption) such as ezetimibe, or proprotein convertase subtilisin/kexin type 9 (PCSK9) inhibitors).

The technical result of the claimed invention consists in reducing interstitial swelling, improving interstitial transport efficiency, reducing atherosclerotic plaques, and reducing stenosis of the affected artery.

The invention relates to an injectable drug composition for the treatment of vascular atherosclerosis, prepared ex tempore, comprising riboflavin, nicotinamide, succinic acid, an anesthetic, and a solvent, characterized in that the anesthetic is 2% lidocaine, and the solvent is 0.9% NaCl.

In one embodiment of the invention, the injectable drug composition comprises the following amounts of components: riboflavin (vitamin B2) 5 mg; nicotinamide (vitamin PP) 25 mg; succinic acid 300 mg; an anesthetic—2% lidocaine 2.0 ml; and a solvent—0.9% NaCl up to 10.0 ml.

In one embodiment of the invention, the drug composition further comprises an enzyme, hyaluronidase, in an amount of 4 U.

In one embodiment of the invention, the drug composition further comprises a metabolic agent, meldonium dihydrate, in an amount of 500 mg.

The invention also relates to the use of the drug composition for the treatment of atherosclerosis of arteries with different localization of lesions.

The invention further relates to the use of the injectable drug composition in the manufacture of a medicinal product for the treatment of vascular atherosclerosis, for anti-inflammatory therapy, for reducing lipid peroxidation, for enhancing lymphatic drainage from the arterial wall in atherosclerosis, and for reducing the size of atherosclerotic plaques and the thickness of the intima-media complex.

The invention also relates to a method for administration of the injectable drug composition in the treatment of atherosclerosis of arteries with different localization of lesions, wherein the injectable drug composition is administered lymphotropically by puncturing the interstitium of the lymphatic region of the artery affected by atherosclerosis, and administration is carried using a syringe dispenser for 30-60 minutes.

The invention also relates to a method of use of the injectable drug composition, wherein the injectable drug composition is administered lymphotropically by puncturing the interstitium of a lymphatic region of the artery affected by atherosclerosis, and administration is carried out using a syringe dispenser for 30-60 minutes, thereby creating a depot of the drug composition with a required concentration of active substances directly in the area of the affected arterial segment, which ensures reduction of atherosclerotic plaques therein.

In carrying out the method of use of the injectable drug composition, administration of the drug composition is performed once daily for 10 to 15 days.

The invention further relates to a method of treating atherosclerosis of arteries, comprising lymphotropic intestinal injection of the injectable drug composition into the lymphatic region of the artery affected by atherosclerosis, by slow administration of the drug composition using a syringe dispenser, in a course of 10-15 days with single daily administration.

In one embodiment of the invention, namely in the treatment of atherosclerosis of mediastinal vessels and cervical vessels, puncture of the interstitium of the lymphatic region (LT3) is performed by inserting a needle through the jugular fossa pretracheally into the previsceral space (5), passing the needle through the superficial lamina (lamina superficialis) (3) and the pretracheal lamina (lamina pretrachealis) (4) of the proper cervical fascia.

In one embodiment of the invention, namely in the treatment of atherosclerosis of cervical vessels, puncture of the interstitium of the lymphatic region (LT4) is performed by inserting a needle through the supraclavicular fossa into the region of the omoclavicular triangle of the neck, passing the needle through the superficial and proper cervical fascia into the interstitium of the previsceral cellular space (5).

In one embodiment of the invention, namely in the treatment of atherosclerosis in the femoral artery, puncture of the interstitium of the lymphatic region (LT10) is performed by inserting a needle into the interstitium of the cellular space of the femoral triangle.

In one embodiment of the invention, namely in the treatment of atherosclerosis of arteries in the pelvic region, puncture of the interstitium of the lymphatic region (LT5 or LT6) is performed by inserting a needle either into the interstitium of the inguinal region above the inguinal ligament (12), or into the interstitium of the inguinal region below the inguinal ligament (12).

In one embodiment of the invention, namely in the treatment of atherosclerosis of the popliteal artery, puncture of the interstitium of the lymphatic region (LT11) is performed by inserting a needle through the popliteal fossa into the interstitium of the cellular space of the popliteal fossa.

In one embodiment of the invention, namely in the treatment of atherosclerosis of the anterior tibial artery, puncture of the interstitium of the lymphatic region (LT12) in the lower third of the leg and of the dorsalis pedis artery is performed by inserting a needle into the interstitium of the cellular space between the tendons of the extensor digitorum longus (24) and the tibialis anterior muscle (23).

In one embodiment of the invention, namely in the treatment of atherosclerosis of the posterior tibial artery, puncture of the interstitium of the lymphatic region (LT13) in the lower third of the leg is performed by inserting a needle into the interstitium of the cellular space posterior to the medial malleolus (22), between the tendons of the flexor digitorum longus (20) and the flexor hallucis longus (21).

### BRIEF DESCRIPTION OF THE DRAWINGS

The present invention is illustrated by the following drawings:

FIG. 1 shows the puncture site of the interstitium of lymphatic region LT3 in the treatment of atherosclerosis of mediastinal vessels and cervical vessels. Anterior view.

FIG. 2 shows the puncture site of the interstitium of lymphatic region LT3 in the treatment of atherosclerosis of mediastinal vessels and cervical vessels. Horizontal plane view.

FIG. 3 shows the puncture site of the interstitium of lymphatic region LT4 in the treatment of atherosclerosis of cervical vessels. Lateral view.

FIG. 4 shows the puncture site of the interstitium of lymphatic region LT4 in the treatment of atherosclerosis of cervical vessels. Horizontal plane view.

FIG. 5 shows the puncture site of the interstitium of lymphatic regions LT5, LT6, and LT7 in the treatment of atherosclerosis of arteries in the pelvic region.

FIG. 6 shows the puncture site of the interstitium of lymphatic region LT10 in the treatment of atherosclerosis of the femoral artery.

FIG. 7 shows the puncture site of the interstitium of lymphatic region LT11 in the treatment of atherosclerosis of the popliteal artery.

FIG. 8 shows the puncture site of the interstitium of lymphatic region LT12 in the treatment of atherosclerosis of the anterior tibial artery in the lower third of the leg and the dorsalis pedis artery, and of lymphatic region LT13 in the treatment of atherosclerosis of the posterior tibial artery in the lower third of the leg.

In the drawings, the following reference numerals are used:
1. Sternocleidomastoid muscle (m. sternocleidomastoideus)
2. Clavicle (os clavicular)
3. Superficial lamina of the proper cervical fascia (lamina superficialis fasciale colli profunda)
4. Pretracheal lamina of the proper cervical fascia (lamina pretrachealis fasciae colli profunda)
5. Pretracheal space (spatium pretracheale)
6. Omohyoid muscle (m. omohyoideus)
7. Carotid sheath (vagina corotica)
8. Prevertebral lamina (lamina prevertebralis)
9. Posterior margin of the sternocleidomastoid muscle (m. sternocleidomastoideus)
10. Inferior belly of the omohyoid muscle (m. omohyoideus)
11. Trapezius muscle (m. trapezius)
12. Inguinal (Poupart's) ligament (l. inguinalis)
13. Superior margin of the pubic region (region pubis)
14. Sartorius muscle (m. sartorius)
15. Adductor minimus muscle (m. adductor minimus)
16. Semimembranosus muscle (m. semimembranosus)
17. Long head of the biceps femoris muscle (caput longus m. biceps femoris)
18. Medial head of the gastrocnemius muscle (caput mediale m. gastrocnemii)
19. Lateral head of the gastrocnemius muscle (caput laterale m. gastrocnemii)
20. Flexor digitorum longus muscle (m. flexor digitorum longus)
21. Flexor hallucis longus muscle (m. flexor hallucis longus)
22. Medial malleolus (malleolus medialis) of the tibia (os tibia)
23. Tibialis anterior muscle (m. tibialis anterior)
24. Extensor digitorum longus muscle (m. extensor digitorum longus)
25. Flexor retinaculum (retinaculum mm. flexorum).

### DETAILED DESCRIPTION OF THE INVENTION

In the present invention, the technical result is achieved by using an injectable drug composition comprising nicotinamide, succinic acid, riboflavin, an anesthetic, and a solvent, wherein the anesthetic is 2% lidocaine, and the solvent is 0.9% NaCl. In addition, the drug composition can further comprise an enzyme, hyaluronidase, and can further comprise a metabolic agent, meldonium dihydrate.

The drug composition is prepared ex tempore.

The quantitative composition of the injectable drug composition is as follows:
Vitamin—riboflavin (vitamin B2), 5 mg;
Vitamin—nicotinamide (vitamin PP), 25 mg;
Metabolic agent—succinic acid, 300 mg;
Anesthetic—2% lidocaine, 2.0 ml;
Solvent—0.9% NaCl, up to 10.0 ml;
Enzyme (optional component)—hyaluronidase, 4 U;
Metabolic agent (optional component)—meldonium dihydrate, 500 mg.

The use of 2% lidocaine as the anesthetic (instead of 0.5% Novocaine) provides a more pronounced anesthetic effect with a smaller volume of the injected medicinal substance and a reduced risk of adverse effects, which enables more reliable repetition of injections and achievement of the technical result.

The use of the optional component hyaluronidase modifies the state of the connective tissue matrix and improves the conductivity of the medicinal substances through the interstitium in a zone of a lymphatic region, making lymphotropic administration more effective in patients with increased connective tissue density.

The use of meldonium dihydrate supplements the metabolic action of succinic acid and enhances the effect in patients with a more advanced stage of the disease, in whom administration of meldonium is not contraindicated.

The technical result is also achieved by administering the drug composition lymphotropically and interstitially by lymphotropic interstitial injection into the lymphatic region where the arterial segment affected by atherosclerosis is localized. Administration of the drug composition is carried out using a syringe dispenser over 30-60 minutes.

Lymphotropic interstitial injections are performed once daily for 10-15 days.

Specifying zones for administration of the medicinal substances makes it possible to create a depot of the drug composition with a required concentration of active substances directly in the area of the affected arterial segment and in the corresponding lymphatic region, thereby achieving the technical result, namely reduction of atherosclerotic plaques.

The use of a syringe dispenser enables slow administration of the drug composition, avoiding formation of large infiltrates, excessive interstitial swelling, and blockage of interstitial transport, and allows repeated use of the same administration zone to maintain the required concentration for 10-15 days and to achieve the therapeutic effect.

The method is carried out as follows:

1. The above-described drug composition is prepared ex tempore as a mixture of medicinal substances comprising vitamins (riboflavin in an amount of 5 mg and nicotinamide in an amount of 25 mg), a metabolic agent (succinic acid in an amount of 300 mg, and, optionally, meldonium dihydrate in an amount of 500 mg), an anesthetic (2% lidocaine 2.0 ml), a solvent (0.9% NaCl 10.0 ml), and, optionally, an enzyme (hyaluronidase 4 U).

2. A lymphotropic interstitial injection of the drug composition is performed by puncturing the interstitium of a lymphatic region depending on a zone of the artery affected by atherosclerosis, followed by administration of the drug composition.

3. The drug composition is administered using a syringe dispenser over 30-60 minutes.

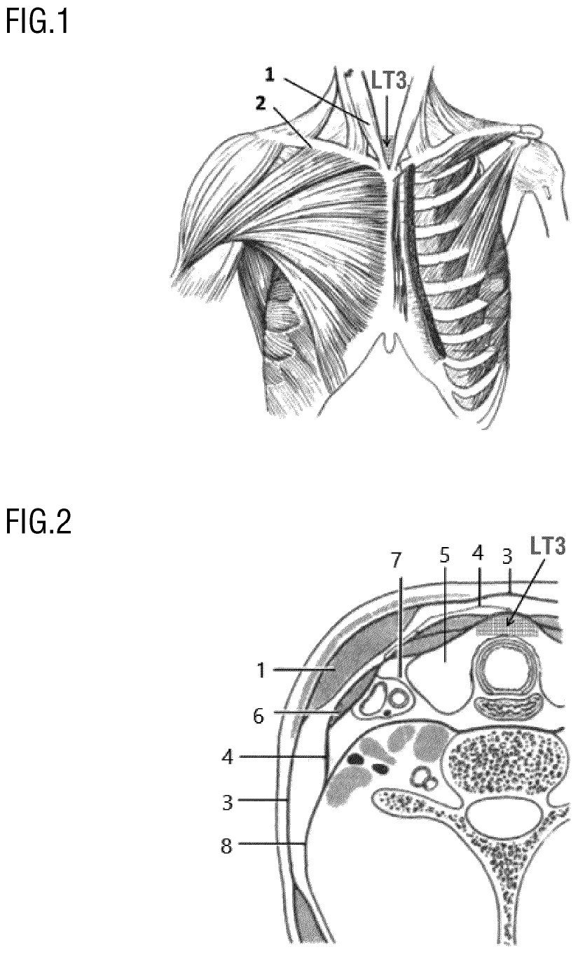

4. In the treatment of atherosclerosis of mediastinal vessels and cervical vessels (lymphatic region LT3 in FIGS. 1 and 2), puncture of the interstitium of the lymphatic region is performed by inserting a needle through the jugular fossa pretracheally into the previsceral space, passing the needle through the superficial lamina (lamina superficialis) (3) and the pretracheal lamina (lamina pretrachealis) (4) of the proper cervical fascia.

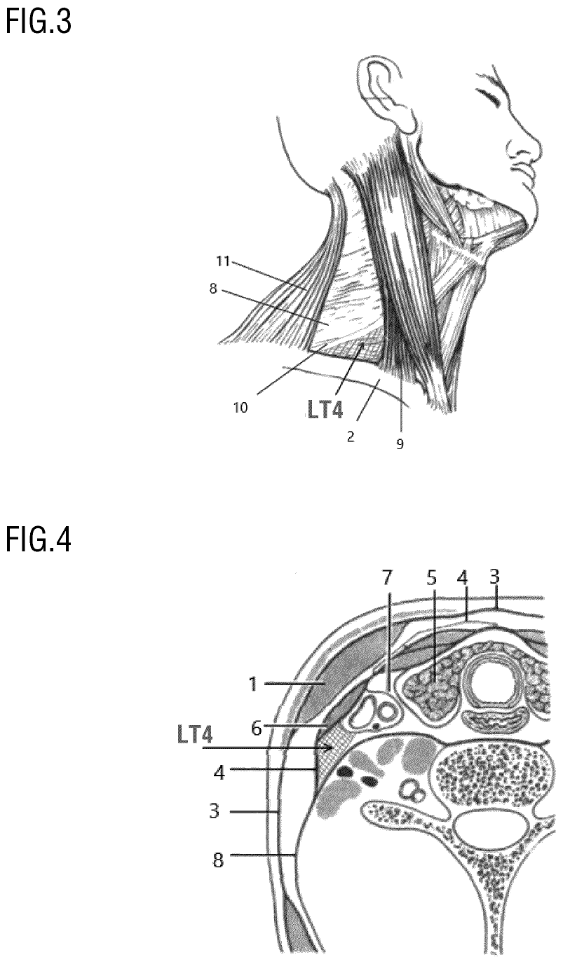

5. In the treatment of atherosclerosis of cervical vessels (lymphatic region LT4 in FIGS. 3 and 4), puncture of the interstitium of the lymphatic region is performed by inserting a needle through the supraclavicular fossa into the omoclavicular triangle of the neck, passing the needle through the superficial and proper cervical fasciae into the interstitium of the previsceral cellular space.

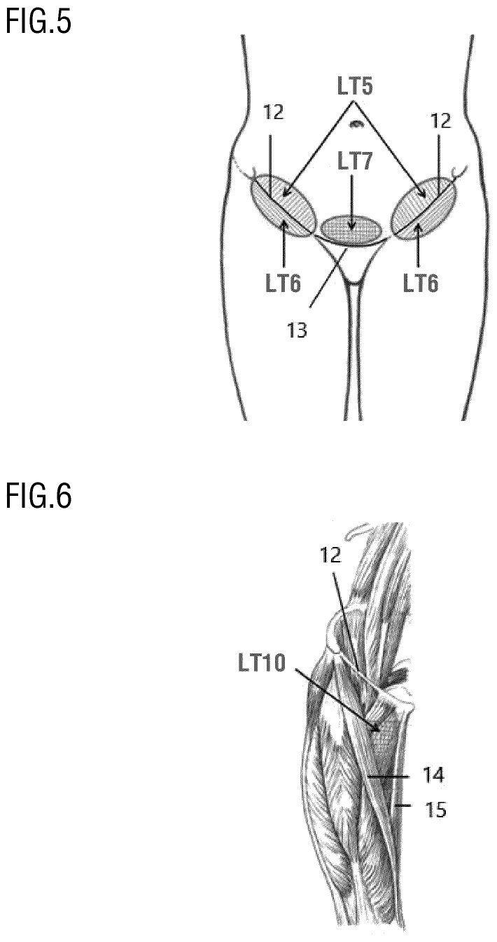

6. In the treatment of atherosclerosis of the femoral artery (lymphatic region LT10 in FIG. 6), puncture of the interstitium of the lymphatic region is performed by inserting a needle into the interstitium of the cellular space of the femoral triangle.

7. In the treatment of atherosclerosis of arteries in the pelvic region (lymphatic region LT5 or LT6 in FIG. 5), puncture of the interstitium of the lymphatic region is performed by inserting a needle either into the interstitium of the inguinal region above the inguinal ligament (12) (puncture LT5) or into the interstitium of the inguinal region below the inguinal ligament (12) (puncture LT6).

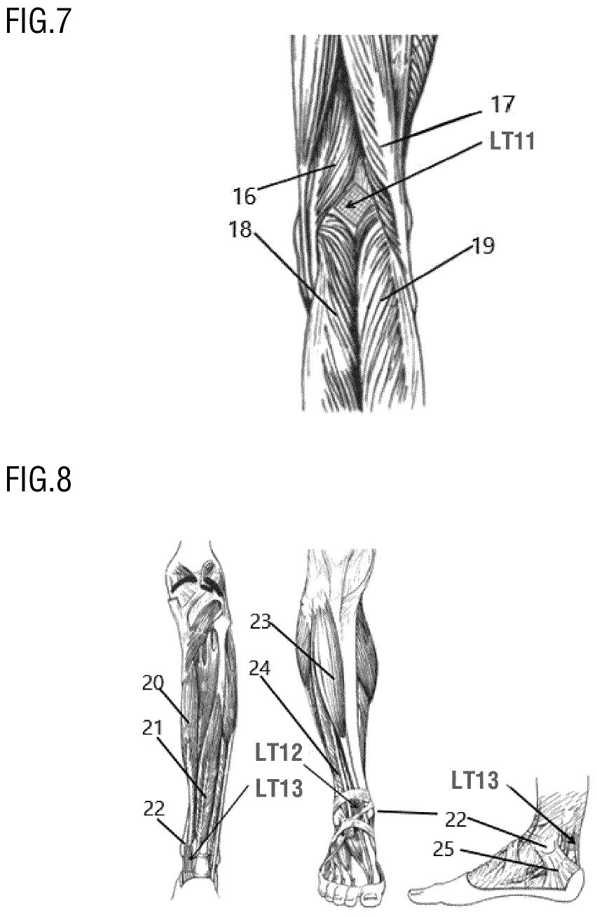

8. In the treatment of atherosclerosis of the popliteal artery (lymphatic region LT11 in FIG. 7), puncture of the interstitium of the lymphatic region is performed by inserting a needle through the popliteal fossa into the interstitium of the cellular space of the popliteal fossa.

9. In the treatment of atherosclerosis of the anterior tibial artery in the lower third of the leg and the dorsalis pedis artery (lymphatic region LT12 in FIG. 8), puncture of the interstitium of the lymphatic region is performed by inserting a needle into the interstitium of the cellular space between the tendons of the extensor digitorum longus (24) and the tibialis anterior muscle (23).

10. In the treatment of atherosclerosis of the posterior tibial artery in the lower third of the leg (lymphatic region LT13 in FIG. 8), puncture of the interstitium of the lymphatic region is performed by inserting a needle into the interstitium of the cellular space posterior to the medial malleolus (22), between the tendons of the flexor digitorum longus (20) and the flexor hallucis longus (21).

11. Lymphotropic interstitial injections are performed once daily for a period of 10-15 days.

### IMPLEMENTATION OF THE INVENTION

The invention is illustrated by the following examples of implementation.

Example 1. Use of the drug composition in atherosclerosis of brachiocephalic and coronary arteries.

Patient M., male, 45 years, presented with complaints of fatigue, elevated blood pressure, headache, tinnitus, and retrosternal pain during physical exertion. The patient had a history of treatment with simvastatin, which was discontinued due to side effects.

Examination performed on 17.12.2017:
1. Ultrasound Doppler of the brachiocephalic arteries revealed stenosis of the common carotid artery (CCA) of 51% on the left and 40% on the right;
2. Multislice computed tomography (MSCT) of the heart revealed stenosis of the posterior descending artery (PDA), diagonal branch (D2), 72% by diameter;
3. 24-hour Holter monitoring of electrocardiography (ECG) and blood pressure (BP).

Diagnosis: ischemic heart disease; atherosclerosis of the brachiocephalic arteries; atherosclerosis of the coronary arteries.

For treatment, along with antihypertensive therapy, the claimed injectable drug composition and the claimed method of administration by lymphotropic interstitial injection were used.

The drug composition comprising riboflavin 5 mg, nicotinamide 25 mg, succinic acid 300 mg, 2% lidocaine 2.0 ml, 0.9% NaCl 10.0 ml, prepared ex tempore in one syringe, was administered as a lymphotropic interstitial injection by puncturing the interstitium of lymphatic region LT4, wherein the drug composition was administered using a syringe dispenser over 30 minutes. Injections of the drug composition were administered once daily for a 10-day course. Courses were repeated four times over a two-year period.

No adverse effects were observed.

Clinical response after the first course of treatment consisted in disappearance of headache and tinnitus and significant reduction of ischemic chest pain. After the second course of treatment, complete resolution of the reported symptoms was achieved, including retrosternal pain.

Follow-up examination in September 2019: MSCT data showed reduction of PDA D2 ostial stenosis from 73 to 29% by diameter and from 93% to 51% by area. Ultrasound Doppler data showed reduction of CCA stenosis from 51% to 10% on the left and from 40% to 15% on the right.

Example 2. Use of the drug composition in atherosclerosis of the vessels of the lower extremities.

Patient V., male, 66 years, presented with complaints of intermittent claudication, leg pain (more severe on the right), nocturnal calf cramps, coldness of the toes, and pain-free walking distance limited to 100 m, with worsening over the last year.

The patient's history included treatment with rosuvastatin 10 mg, discontinued due to side effects (gastralgia, diarrhea, dizziness), coronary artery bypass grafting of 4 vessels, and stenting of the common iliac artery in 2010.

Ultrasound Doppler of the leg arteries performed on 09.12.2021 revealed thickened arterial walls (intima-media complex up to 1.6 mm, calcified), heterogeneous atherosclerotic plaques of the common femoral and superficial femoral arteries bilaterally, with stenosis reaching up to 78% in various sites. Data on stenosis are presented in Table 1.

TABLE 1

| | |
|---|---|
| Sep. 12, 2019 | Oct. 3, 2020 |

Right side

| | | |
|---|---|---|
| Common femoral artery, stenosis | 47% | 41% |
| Lower third of the superficial | 78% | 69% |
| femoral artery, stenosis | | |
| Popliteal artery, stenosis | 30% | 23% |

Left side

| | | |
|---|---|---|
| Common femoral artery, stenosis | 43% | 30% |
| Superficial femoral artery, stenosis | 23% | 15% |

Diagnosis: ischemic heart disease (IHD). Condition after coronary artery bypass grafting (CABG), pulmonary artery thromboembolism (PTE), and stenting of the common iliac artery (CIA). Stage III hypertension. Cardiovascular (CVC) risk grade 4. Stenosing atherosclerosis and calcification of the arteries of the lower extremities.

For treatment, together with antihypertensive therapy, the claimed method of administration of the drug composition by lymphotropic interstitial injection was used.

The drug composition comprising riboflavin 5 mg, nicotinamide 25 mg, succinic acid 300 mg, 2% lidocaine 2.0 ml, 0.9% NaCl 10 ml, was administered as a lymphotropic interstitial injection by puncturing the interstitium of lymphatic region LT10, wherein the drug composition was administered using a syringe dispenser over 30 minutes.

The drug composition was administered once daily for a 12-day course. The course was repeated twice over a period of 4 months.

No adverse effects were observed.

The clinical response after the first course of treatment consisted in an increase of pain-free walking distance to 700 m. After the second course of treatment, the patient reported complete resolution of calf pain and the ability to walk for a long time without fatigue.

Follow-up examination on 10.03.2020: ultrasound Doppler data showed decreased echogenicity of atherosclerotic plaques and a reduction of stenosis of the common and superficial femoral arteries, as well as the popliteal artery, by 9-15%. Detailed comparative data are provided in Table 1.

Overall, the claimed drug composition and method of its administration were used in 43 patients. The median reduction of stenosis after one course of treatment was 11% in relative terms. The median number of days of administration of the drug composition per course was 13 injections in the treatment of atherosclerosis of the vessels of the lower extremities and 15 injections in the treatment of atherosclerosis of cervical vessels. Thirty-eight patients reported significant clinical improvement, including improved general well-being and work capacity, reduced weakness and dyspnea, increased exercise tolerance (6-minute walking test), reduced meteorological sensitivity, less frequent fluctuations in blood pressure, and decreased pain syndrome.

### INDUSTRIAL APPLICABILITY

Use of the claimed method provides the following advantages:

1. Increased effectiveness in the treatment of atherosclerotic diseases of cervical vessels, cerebral vessels, coronary vessels, mediastinal vessels, and vessels of the lower extremities, with significant positive dynamics achieved using widely available pharmaceutical agents.

2. A paradigm shift in therapy from treatment of hyperlipidemia to direct treatment of arterial wall inflammation and inadequate lymphatic drainage of oncosis and apoptosis products of the plaque.

3. Prevention of cardiovascular complications without the use of statins and without surgical intervention.

1. A method of manufacturing an ink composition for depositing of a photoactive layer of an optoelectronic device, the method comprising:
providing a first phase liquid comprising a nonpolar solvent with nanoparticles dispersed in the nonpolar solvent, which nanoparticles are capped with a shell of first ligands, the first ligands comprising a surface binding head group and an alkyl or alkenyl tail for, at least initially, keeping the nanoparticles in a colloidal suspension in the nonpolar solvent;
providing a second phase liquid comprising a first polar solvent with second ligands dissolved therein, the second ligands having a molecular architecture including:
a thiol head group for binding to a surface site of said nanoparticle;
an ionically charged tail group having a counter ion associated therewith; and
an alkyl spacer separating the head group and the tail group having a total number of carbon atoms in a range from 1 to 3,
a biphasic ligand exchange process wherein the second ligands displace the first ligands that are bound to the nanoparticles thereby releasing the first ligands to the first phase liquid, the biphasic ligand exchange process comprising contacting the first and second phase liquid to form a dispersion of the nanoparticles that are capped with a shell of the second ligands in the second phase liquid.

2. The method according to claim 1, wherein the second ligands are: 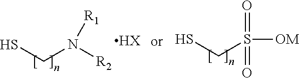
wherein
“n” is between 1 and 3;
“R₁” and “R₂” are each independently selected from H and methyl;
“X” is a halide; and
“M” is an alkali metal.

3. The method according to claim 1, comprising:
separating the nanoparticles that are capped with a shell of the second ligands from the first polar solvent; and
redispersing the separated nanoparticles in a printable liquid medium comprising a second polar solvent.

4. The method according to claim 1, wherein
the first polar solvent comprises N,N-dimethylformamide, N-methylformamide, or a combination thereof; and
the second polar solvent comprises an at least partially fluorinated lower alcohol, or a mixture of water and ethylene glycol, wherein the volume fraction of ethylene glycol in the mixture is in a range between five and fifty percent.

5. The method according to claim 1, comprising dissolving a disulfide moiety into the second phase liquid so as to form the second ligands as dissociation products of the disulfide moiety.

6. The method according to claim 1, the method comprising:
determining an amount of the second ligand that is to be comprised in the first polar solvent relative to an amount needed to completely cap the nanoparticles with a shell of the second ligands; and
providing the second phase liquid comprising the first polar solvent with second ligands in an amount of less than 1.05 times the pre-determined amount.

7. The method according to claim 1, comprising washing the separated nanoparticles with one or more portions of a solvent for the first ligand.

8. The method according to claim 1, comprising adding a third solvent to the first phase liquid and second phase liquid, the third solvent being miscible with both the nonpolar solvent and the first polar solvent.

9. An ink composition for use in the deposition of a photoactive layer of an optoelectronic device, the ink composition comprising:
a colloidal suspension of nanoparticles in a printable liquid medium comprising a polar solvent, wherein the nanoparticles are capped with a shell of second ligands replacing an initial shell of different first ligands, wherein the second ligands have a molecular architecture including:
a thiol head group bound to a surface site of said nanoparticle;
an ionically charged tail group having a counter ion associated therewith; and
an alkyl spacer separating the head group and the tail group having a total number of carbon atoms n in a range from 1 to 3,
wherein the polar solvent comprises an at least partially fluorinated lower alcohol, or a mixture of water and ethylene glycol, wherein the volume fraction of ethylene glycol in the mixture is in a range between five and fifty percent.

10. The ink composition according to claim 9, wherein the composition is essentially without first ligands and free unbound second ligands.

11. The ink composition according to claim 9, wherein the second ligands are: 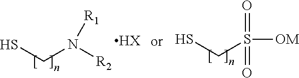
wherein
“n” is between 1 and 3;
“R₁” and “R₂” are each independently selected from H and methyl;
“X” is a halide; and
“M” is an alkali metal.

12. The ink composition according to claim 9, comprising a pyrrolidone additive.

13. The ink composition according to claim 9, comprising one or more polymeric additives.

14. The ink composition according to claim 9, comprising a p-type dopant or an n-type dopant.

15. A method of manufacturing an imaging device comprising a stack of photoactive layers including at least a p-type photoactive layer and an n-type photoactive layer, the method comprising:
depositing one or more layers of the ink composition according to claim 14 and comprising the p-type dopant; and
depositing one or more layers of the ink composition according to claim 14 and comprising the n-type dopant;
the deposited layers forming a stack of photoactive layers including at least a p-type photoactive layer and an n-type photoactive layer, wherein the photoactive layers are amorphous, comprising a disordered structure of nanoparticles formed by quantum dots resulting from the deposition by applying the respective ink compositions comprising colloidal quantum dots.

16. A dry composition, comprising the nanoparticles that are capped with a shell of second ligand obtainable by
performing the method according to claim 1;
separating the nanoparticles that are capped with a shell of the second ligands from the first polar solvent of the second phase liquid; and
drying the separated nanoparticles.

17. A method for manufacturing a printable ink composition, the method comprising dispersing the dry composition of claim 16 in a polar solvent comprising an at least partially fluorinated lower alcohol, or a mixture of water and ethylene glycol, wherein the volume fraction of ethylene glycol in the mixture is in a range between five and fifty percent.

18. The method of claim 2, wherein X is Cl.

19. The method of claim 2, wherein M is Na or K.

20. The ink composition of claim 11, wherein X is Cl and/or wherein M is Na or K.
